(19) United States
(12) Patent Application Publication
(10) Pub. No.: US 2017/0367579 A1
(43) Pub. Date: Dec. 28, 2017

(54) EMBEDDED BIOSENSORS FOR ANATOMIC POSITIONING AND CONTINUOUS LOCATION TRACKING AND ANALYSIS OF MEDICAL DEVICES

(71) Applicant: Bruce REINER, Berlin, MD (US)

(72) Inventor: Bruce REINER, Berlin, MD (US)

(21) Appl. No.: 15/632,817

(22) Filed: Jun. 26, 2017

Related U.S. Application Data
(60) Provisional application No. 62/355,031, filed on Jun. 27, 2016.

(51) Int. Cl. A61B 5/00; A61B 6/03; A61B 6/00; A61B 17/12

(52) U.S. Cl. CPC A61B 5/0022; A61B 6/501; A61B 6/032; A61B 2560/0271; A61B 17/1214

(57) ABSTRACT

The present invention is directed to a miniaturized biosensor and nanotechnology which is embedded in a variety of medical devices which can be used for real-time device location tracking and analysis, for the purpose of optimizing device positioning both at the time of initial placement and throughout its clinical use (i.e., device continuum). The continuously acquired device-specific standardized data is then transmitted through wireless communication networks to provide continuous feedback and alerts to authorized clinical providers as to device positioning, clinical performance, and presence of pathology.

### CROSS-REFERENCE TO RELATED APPLICATIONS

The present invention claims priority to U.S. Provisional Patent Application No. 62/355,031, filed Jun. 27, 2016, the contents of which are herein incorporated by reference in their entirety.

### BACKGROUND OF THE INVENTION

### 1. Field of the Invention

The present invention is directed to a miniaturized biosensor and nanotechnology which is embedded in a variety of medical devices which can be used for real-time device location tracking and analysis, for the purpose of optimizing device positioning both at the time of initial placement and throughout its clinical use (i.e., device continuum). The continuously acquired device-specific standardized data is then transmitted through wireless communication networks to provide continuous feedback and alerts to authorized clinical providers as to device positioning, clinical performance, and presence of pathology.

### 2. Description of the Related Art

In a related patent U.S. patent application Ser. No. 15/434,783, entitled “Method and Apparatus for Embedded Sensors in Diagnostic and Therapeutic Medical Devices”, filed Feb. 16, 2017, by the same inventor, and which is herein incorporated by reference in its entirety, a “smart” medical technology was disclosed which utilized miniaturized biosensors and nanotechnology embedded within a variety of medical devices for the purpose of creating, recording, and analyzing real-time medical data in vivo. In turn, this standardized data could be recorded in a series of referenceable databases (which are specific to the individual patient, disease state, device (and device manufacturer), institutional provider, and clinical operator), and used to create customizable analytics used for clinical decision support, personalized medicine, establishment of “best practice” guidelines and standards (i.e., evidence-based medicine), comparative technology assessment, and technology optimization specific to the individual attributes of the patient, disease, and operator of record.

While device localization using embedded biosensors can be readily applied to a myriad of medical devices, gastrointestinal feeding tubes are of note since the technical and clinical challenges are fairly straightforward, the utilization rate of these devices is extremely high in conventional practice, the complication rate is well documented, and the existing methods in use are often fraught with diagnostic error, excessive cost, and time delays (which can adversely affect clinical outcomes).

More specifically, nasogastric and gastrostomy tubes are commonly used examples of medical devices routinely used for enteral nutritional support, which plays a fundamental role in the clinical management of patients with poor voluntary oral intake, chronic neurological or mechanical dysphagia, intestinal failure, and the critically ill. These devices used for enteral nutrition can be placed by nasal insertion (e.g., nasogastric tube), guided percutaneous insertion (e.g., percutaneous gastrostomy tube), or surgery (e.g., jejunostomy tube).

The complication rates for these devices has been well documented in the medical literature. Since nasogastric tubes are most commonly blindly inserted (i.e., without supporting guidance techniques), they are frequently malpositioned in the respiratory system, which can have catastrophic results. The malpositioning error rates for nasogastric tubes have been reported to be as high as 20% in adult patients. Further, the small bore silicone nasogastric tubes in common use contain metallic weighted tips and stiffening introductory stylets which create added potential for malpositioning and complications including (but not limited to) pneumothorax, empyema, bronchopleural fistula, mediastinitis, pneumonia, and perforation.

A number of methods and tests are currently used to determine nasogastric tube placement position, the most common of which are radiography (e.g., chest or abdominal x-ray), visual and pH testing of aspirate, and insufflation of air combined with auscultation. All of these tests have their diagnostic limitations and are far from foolproof. At the same time radiography adds time delays, excessive cost, and increasing radiation. An optimal strategy would be to have a single test which could provide accurate and definitive information at the patient's bedside, be independent of operator error, and not introduce increased cost or health risk to the patient.

Equally important to the requirement of accurate localization at the time of device insertion is the challenge of continuously assessing device location over time, since changes in device location are commonplace. Nasogastric and gastrostomy tubes frequently undergo positional change (which is frequently the result of patient or nursing manipulation), and require frequent and repeated position reassessment. This routinely takes the form of ionizing medical imaging studies (e.g., portable radiography with or without contrast injection, CT), which further increases cost, radiation dose, and potential time delays in clinical management. Even with routine radiographic surveillance, small positional changes in tube placement can often go undetected, which may have negative clinical implications. Furthermore, these tests must be actively requested and are not customarily performed on a predictable and continuous basis. When performed, they are often subjected to human error in interpretation; which can lead to catastrophic consequences in the critically ill patient. When not ordered, any resulting positional change in the device will go undetected, which can be equally catastrophic, as in the common encountered example of a dislodged percutaneous gastrostomy tube, with the tip outside of the stomach and in the peritoneal cavity, any injected fluid (e.g., feedings, medication) would flow into the peritoneal cavity and could lead to peritonitis—a frequently fatal condition.

Accordingly, the goal of the present invention is to create technology which can be readily applied and integrated into a wide variety of medical devices utilizing real-time anatomic and physiologic data to optimize device position and functionality, throughout the clinical lifetime of the device.

### SUMMARY OF THE INVENTION

The present invention is directed to a miniaturized biosensor and nanotechnology which is embedded in a variety of medical devices which can be used for real-time device location tracking and analysis, for the purpose of optimizing device positioning both at the time of initial placement and throughout its clinical use (i.e., device continuum). The continuously acquired device-specific standardized data is then transmitted through wireless communication networks to provide continuous feedback and alerts to authorized clinical providers as to device positioning, clinical performance, and presence of pathology.

As multiple device positioning and anatomic data is collected over time within an individual patient, the data collected can be used to create a patient-specific three-dimensional (3D) anatomic roadmap which can provide both an historical record of device placements over the continuum of care, as well as an anatomic reference guide for future device placements. This device-specific anatomic data can be correlated with other anatomic data sources (e.g., cross-sectional medical imaging studies (computed tomography (CT), magnetic resonance imaging (MRI)), operative notes, endoscopy photographic images) to provide multi-source anatomic reference maps which can take into account the unique anatomic (and pathologic) attributes of each individual patient. This can be especially helpful in patients who have anatomic variations due to congenital causes, underlying pathology, or iatrogenic reasons (e.g., prior surgery).

The present invention creates technology which can be readily applied and integrated into a wide variety of medical devices utilizing real-time anatomic and physiologic data to optimize device position and functionality, throughout the clinical lifetime of the device. In addition to integration of this technology into existing medical devices, the technology (and acquired knowledge) of the present invention can be used in the creation of “self-navigating” smart medical devices, which incorporate miniaturized motors and self-propelling technology for optimal positioning which could dramatically reduce (or even eliminate) iatrogenic complications caused by human mechanical or oversight errors.

While device localization using embedded biosensors can be readily applied to a myriad of medical devices, the present invention discloses a gastrointestinal feeding tube as an exemplary embodiment, due to the technical and clinical challenges in the existing art.

In one embodiment, the computer-implemented method of determining medical device positional changes within a body of a patient, includes: providing a medical device for internal use within the body of the patient during a medical procedure, the medical device having a plurality of sensors disposed at predetermined intervals along a length of the medical device; receiving data from the sensors on a position of the medical device in the body of the patient, and recording the data into a database of a computer system, performing an analysis of the data using a processor of the computer system; wherein when the analysis of the data received from the sensors indicate a positional change of the medical device, issuing an alert that the medical device has changed its position.

In one embodiment, the predetermined intervals include a device origination point, a device termination point, and transition points which indicate anatomical transition points.

In one embodiment, sensors are disposed in at least one of outer walls or inner walls of the medical device.

In one embodiment, the sensors include one or more types of sensors or biomarkers, including at least one of electrical sensors, chemical sensors, ultrasound sensors, motion sensors, or pressure sensors.

In one embodiment, the sensors measure at least one of pH, oxygen, carbon dioxide, radiation, curvature, coiling, motion, pressure, sound, flow volume, velocity and directionality, fluid characteristics, cellularity, or size.

In one embodiment, the anatomical transition points are fixed.

In one embodiment, the position markers for the anatomical transition points are correlated with the position markers for physiologic transition points, to provide accuracy in device localization.

In one embodiment, the data is transmitted continuously by the sensors.

In one embodiment, the alert is issued by electronic methods.

In one embodiment, the data is synchronized with other anatomic data to create a patient-specific anatomic reference map.

In one embodiment, the data from the patient-specific anatomic reference map is incorporated into the medical device prior to placement to provide visual or auditory feedback.

In one embodiment, the method further includes: synchronizing the sensors and the data from the patient-specific anatomic reference map to make real-time modifications to said patient-specific anatomic reference map.

In one embodiment, the method further includes: correlating the data from the patient specific anatomic reference map with device-specific sensor roadmaps, to provide an anatomic reference point for each of the sensors contained within the medical device.

In one embodiment, the method further includes: combining and analyzing data from multiple patients, device categories, individual healthcare or institutional providers, or device manufactures.

In one embodiment, each of the sensors emit a characteristic signal to identify its specific location on the medical device; and the signal is correlated with a device specific roadmap.

In one embodiment, the method further includes: providing a graphical display of said positional change of the medical device.

Thus, has been outlined, some features consistent with the present invention in order that the detailed description thereof that follows may be better understood, and in order that the present contribution to the art may be better appreciated. There are, of course, additional features consistent with the present invention that will be described below and which will form the subject matter of the claims appended hereto.

In this respect, before explaining at least one embodiment consistent with the present invention in detail, it is to be understood that the invention is not limited in its application to the details of construction and to the arrangements of the components set forth in the following description or illustrated in the drawings. Methods and apparatuses consistent with the present invention are capable of other embodiments and of being practiced and carried out in various ways. Also, it is to be understood that the phraseology and terminology employed herein, as well as the abstract included below, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the methods and apparatuses consistent with the present invention.

### BRIEF DESCRIPTION OF THE DRAWINGS

FIG. 1 is a schematic diagram of a side view and end view of a medical device with embedded sensors in its outer and inner walls and tip, according to one embodiment consistent with the present invention.

FIG. 2 is a schematic diagram of a side view and end view of a medical device, showing a variety of sensors which can be disposed on the medical device, according to one embodiment consistent with the present invention.

### DESCRIPTION OF THE INVENTION

The present invention is directed to a miniaturized biosensor and nanotechnology which is embedded in a variety of medical devices which can be used for real-time device location tracking and analysis, for the purpose of optimizing device positioning both at the time of initial placement and throughout its clinical use (i.e., device continuum). The continuously acquired device-specific standardized data is then transmitted through wireless communication networks to provide continuous feedback and alerts to authorized clinical providers as to device positioning, clinical performance, and presence of pathology.

The present invention is designed to address the existing deficiencies in medical devices by accurately assessing device position both at the time of insertion and throughout its clinical use through continuous recording of objective data measurements; with the ability to perform longitudinal data analysis for identifying small and often undetected change in device location and functionality. While the specific types of embedded biosensors may differ in accordance with the individual medical device, its designed clinical purpose, and the organ system in which it resides, the overall technical and clinical strategy remains the same. The ability to collect and report these data prospectively and in real time, provides a method of immediate intervention in the event of unexpected location or positional change, while using computerized methods of data analysis to avoid human oversight or error.

Real-Time Medical Device Location Guidance

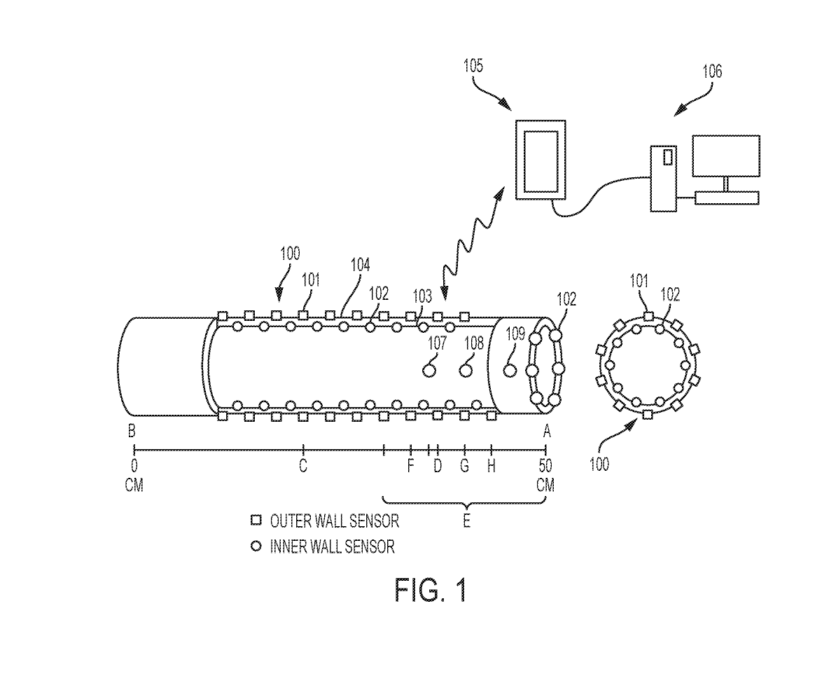

In one exemplary embodiment of the present invention, the functionality involves the embedding of biosensors 101, 102 (see FIG. 1) within the medical device 100 (i.e., catheter) which measures a variety of physiologic (or pathologic) parameters which can provide information relating to the anatomic location of the device 100 at any single point in time. (See U.S. patent application Ser. No. 15/434,783, filed Feb. 16, 2017, and U.S. patent application Ser. No. 15/257,208, filed Sep. 6, 2016, by the same inventor, the contents of both of which are herein incorporated by reference in their entirety).

The sensors 101, 102 can be embedded within any of the inner walls 103 or outer walls 104 of the device, at a predefined location, and the sensors 102, 103 are integrated with a computer device (i.e., smartphone 105, computer system 106), which records data from the medical device 100 in a device specific roadmap. The computer system 106 includes standard computer technology, such as a display, input mechanism (i.e., keyboard, mouse), and microprocessor which runs a program, and a memory in which a database of information is stored. The computer system 106 may be hand-held, or may use both a hand-held and client computer and/or server, and may be wirelessly or hardwired to the medical device 100.

The roadmap created by the computer system 106 defines the exact location, functionality, and specific data attributed to each individual biosensor 101, 102 (or related nanotechnology) embedded within the individual medical device 100. The specific types of biosensors 101, 102 deployed in each medical device 100 are defined by the organ system in which it resides, the method (and course) of device placement, device functionality, and the specific data-derived device parameters being analyzed. Further, whether outer wall 101 or inner wall sensors 102 or both are necessary, will depend on those same parameters.

In one exemplary embodiment of medical devices used for enteral nutritional support, the primary functionality of the device 100 includes delivery of nutritional supplements and medication for absorption within the gastrointestinal tract. While the gastrointestinal tract is the primary organ system of record, the desired anatomic region of interest varies in accordance with the specific device being used. Desired anatomic locations may include the stomach, duodenum, jejunum (proximal small bowel), or ileum (distal small bowel). The expected anatomic structures in which the device 100 will pass through will in large part be determined by the method and course of device 100 placement.

In one exemplary embodiment of a nasogastric tube 100 terminating in the duodenum and inserted in the nasal cavity, the expected course of the device 100 will include the nasal cavity, nasopharynx, esophagus, stomach, and duodenum. While identifying the termination point of the device 100 is of highest interest, additional data charting the full course and anatomic location data throughout the entire length of the device 100 is beneficial; especially when device-related complications occur and specific localization assists in treatment planning and potential intervention. In order to accomplish this task of complete device 100 localization, biosensors 101, 102 can be embedded throughout the entire length of the device 100, at predefined intervals. The data derived from these embedded biosensors 101, 102 can be analyzed by the program and will assist in defining device 100 location, positional changes over time, and the specific location of device 100 malfunction and/or pathology.

In one embodiment, the specific types of data used for device 100 location tracking may vary in accordance with device 100 location and functionality. In the example of the nasogastric tube, biosensors 101, 102 used for location tracking may include (but are not limited to) biosensors 101, 102 measuring pH, carbon dioxide, and internal flow rates. The pH sensor is especially useful for it provides an important method of determining the anatomic transition points of the nasogastric tube as it passes between the esophagus, stomach, and duodenum. While esophageal pH measures tend to be alkaline (in the absence of gastroesophageal reflux) due to the presence of swallowed saliva, upon entering the stomach, the pH measures will dramatically shift due to highly acidic contents within the stomach. Subsequently, as the nasogastric tube (and its embedded biosensors 101, 102) pass from the distal stomach into the duodenum, the pH will significantly rise again to more alkaline levels. By embedding pH sensors 101, 102 into the nasogastric tube walls 103, 104 throughout its length the specific anatomic transition points between the esophagus, stomach, and duodenum can be defined, as well as the specific locations of the device 100 within each individual anatomic structure.

In one exemplary embodiment, if the device 100 (i.e., nasogastric tube) measures 50 cm in length and has embedded pH biosensors 101, 102 at 5 cm intervals from the proximal to distal tips of the device 100, then a total of 11 individual pH biosensors will be present. If the biosensor 101, 102 at position 30 cm of the device (i.e., 30 cm from the proximal-most tip of the device 100) records a dramatic drop in pH, then one can assume that this is the transition point at which the device 100 has passed from the distal esophagus into the proximal stomach (which is anatomically referred to as the gastric cardia). Now if another transition zone is recorded (from acidic to alkaline pH) by the biosensor 101, 102 at position 45 cm of the device 100, then this would be the expected location of the proximal duodenum (i.e., duodenal bulb). If the specific device 100 contains three individual side holes 107, 108, 109 (used as accessory pathways for fluid passage, apart from the distal tube tip) at predefined locations, then biosensors 101, 102 at those positions could also record data which is used for the collective biosensor data and device 100 roadmap.

Accordingly, in one exemplary embodiment, the net anatomic positioning of the device 100 throughout its length is as follows:

A. Device Termination Point (Device Position 50 cm): Proximal Duodenum.

B. Device Origination Point (Device Positon 0 cm): Right Nasal Cavity.

C. Transition Point #1 Esophagus to Gastric Cardia: Device Position 30 cm.

D. Transition Point #2 Gastric Antrum to Duodenal Bulb: Device Position: 45 cm.

E. Length of Gastric Lumen: 15 cm.

F. Location of Device Side hole 107: Device Position 44 cm.

G. Location of Device Side hole 108: Device Position 46 cm.

H. Location Device Side hole 109: Device Position 48 cm.

Based upon this data, a user can conclude that the first side hole 107 is in the distal most portion of the gastric antrum (i.e., distal stomach), the second side hole 108 is in the proximal duodenal bulb, and the third side hole 109 is in the distal duodenal bulb. Since the length of the gastric lumen is 15 cm (based on the two defined transition point locations), one can surmise that the stomach is in a state of relative decompression (if distended, one would expect a much greater length, on the order to >25 cm).

Over time, if the device 100 was to physically move (or if the stomach was to become distended), the positioning of the device embedded biosensors 101, 102 would likely change and this would be reflected by changes in measured pH at specific biosensor locations. By having the ability to continuously measure pH throughout predefined locations of the device 100, a medical professional can be alerted to subtle positional changes in the device 100 which would likely go undetected through conventional means.

In one embodiment, in addition to utilizing pH measuring biosensors 101, 102 for locational assessment; other types of biosensors may prove useful and provide synergistic data for device positional assessment. Knowing that nasogastric tubes are frequently malpositioned in the respiratory system, accurate detection requires biosensors 101, 102 which can provide measurements specific to the anatomy of concern. In this case, since gas exchange is the principle function of the respiratory system, a logical biosensor measurement would include carbon dioxide levels (which would be expected to be high in the trachea and bronchi and almost nonexistent in the gastrointestinal system).

This capability highlights one of the unique attributes of the present invention, which is, whenever possible, to obtain multiple measurements which can be recorded in the database, where the program can analyze physiology specific to different anatomic structures or organ systems. This represents both the desired anatomic location of the medical device 100, as well as a commonly encountered “malpositioned device” anatomic location. In the above example of the nasogastric tube, the desired organ system is the gastrointestinal tract (i.e., pH measures) and the undesired (or malpositioned) organ system is the respiratory tract (i.e., carbon dioxide measures).

In the commonly encountered (and dangerous) situation where the nasogastric tube is improperly positioned in the respiratory tract, the carbon dioxide biosensors 101, 102 will record unexpectedly high levels of carbon dioxide which will cause the program of the computer system to trigger an automated alert to the clinical provider by electronic methods, such as a warning on the computer system, an email, text, telephone, facsimile, etc., notifying them of the improper device 100 positioning. The recordation of synchronous measures throughout a wide array of biosensor locations will provide further evidence of device malpositioning, as analyzed by the program. Having two different types of biosensors 101, 102 obtaining simultaneous measurements will further strengthen the accuracy of device 100 location tracking. Knowing that routine drops in pH typically occur at device 100 positions of 30 cm+/−5 cm; the absence of acidic pH recordings at this level will provide additional verification of the malpositioned device in the respiratory tract.

In one embodiment, another type of sensor 101, 102 which can be incorporated into the medial device 100 to improve device placement and anatomic positioning are sensors 101, 102 designed to detect curvature (i.e., as opposed to straight line) in the course of device 100 passage. It is fairly common for certain types of flexible (i.e., non-rigid) devices to often demonstrate curvature in their course or even outright coiling, which may serve to limit device positioning and functionality.

In one example, a nasogastric tube 100 may coil in an unintended location (e.g., distal esophagus, hiatal hernia, proximal stomach) and prevent distal passage of the tube 100 into its desired final position. Another commonly encountered example is that of a small bore vascular catheter 100 (e.g., PICC line) which is inserted from a peripheral location (e.g., arm), with the intended goal of terminating in a central location (e.g., superior vena cava, right atrium) for the intended purpose of drug delivery. The flexibility and relatively small size of this catheter often results in the catheter migrating out of a larger vein and into a small venous tributary, which diminishes functionality and places the patient at greater risk for developing a device related complication (e.g., venous thrombosis).

By inserting sensors 101, 102 into the device walls 103, 104 which track device 100 directionality and deviation from a straight-line course (relative to adjacent sensors), one can create a smart device 100 capable of providing real time feedback to the operator, of suboptimal device location/positioning and requirement for repositioning. In current clinical practice, the identification of device curvature, coiling, or migration into secondary (undesired) anatomic structures requires medical imaging techniques (e.g., x-ray, CT, venography), which delays clinical management/treatment, incurs additional and unwanted radiation exposure to the patient, and increases to cost for healthcare delivery. Often, these limitations in device positioning go unnoticed or are left alone (even when documented), placing the patient at increased risk for device related complication or decreased functionality.

In one embodiment, the present invention can be used to provide data related to an abnormal course of the medical device 100, and the ability to record and monitor the distance between adjacent biosensors 101, 102. When a device 100 is following a straight-line course, this inter-sensor distance remains constant and is consistent with the schematics contained within the device sensor roadmap. However, when the device 100 pathway deviates from a straight-line path through abnormal curvature, coiling, or kinking, the measured inter-sensor distance decreases for those sensors 101, 102 associated with the coiled or kinked portion of the device 100. When these localized changes in inter-sensor distances are correlated by the program of the computer system, with the device roadmap, one can effectively create a three-dimensional (3-D) representation of the abnormality in device positioning. As manipulation of the device 100 is performed, the continuous analysis by the program of the involved inter-sensor distances can assist the operator in correcting the course and positioning of the device 100. Commonly encountered instances where devices become kinked, coiled, or follow an abnormal curved pathway include (but are not limited to) vascular catheters coursing into small branch vessels, a nasogastric tube coiling in a hiatal hernia, or proximal stomach, and kinking of a percutaneous drainage catheter. Thus, one can see how coiling of a nasogastric tube will produce localized alterations in the inter-sensor distances, when compared with those same distances within a normally positioned nasogastric tube, which follows a straight-line course.

While the above example of a nasogastric tube is described with respect to the present invention, the same concepts can be applied to a myriad of medical devices, encompassing essentially all organ systems and anatomy, along with a broad spectrum of disease processes. Table 1 lists numerous examples of medical devices and the associated biosensors which are pertinent to their initial localization and continuous position monitoring.

TABLE 1
Examples of Device Specific Biosensors for Optimizing Device
Positioning and Continuous Device Location Tracking

| Type of Biosensor | Medical Device | Anatomy/Organ System |
|---|---|---|
| pH | Nasogastric tube | Gastrointestinal |
| | Gastrostomy tube | |
| O₂ (oxygen), CO₂ | Endotracheal tube | Respiratory/Pulmonary |
| (carbondioxide) | | |
| Vibroacoustic | Thoracostomy tube | |
| Organic Solutes | Bladder Catheter | Genitourinary |
| (Urea, Creatinine) | Nephrostomy tube | |
| Electrical | Pacemaker | Cardiac |
| | AICD | |
| Protein | Ventriculostomy | Central Nervous System |
| Ions | VP Shunt Catheter | |
| Flow, Pressure | Venous Catheter | Vascular |
| | Arterial Stent Graft | |
| Bilirubin, | Biliary Catheter | Hepatobiliary |
| Bile Acids | Cholecystectomy Tube | |

The present invention is directed to a number of unique features or embodiments, that apply to all the disclosed sensor-guided devices, regardless of device type and organ system. In one embodiment of the present invention, the sensor-derived data is objective, dynamic (i.e., continuously updated), and directly communicated to the operator during the course of device placement. This latter attribute provides a method for refining and modifying device placement during the course of the procedure, without having to use ancillary “after the fact” tests (e.g., medical imaging exams) to verify device placement and modify as needed.

In another embodiment, the biosensor derived data, analysis, and subsequent actions are the result of at least two different kinds of sensors 101, 102, thereby eliminating the reliance on a single device, whose data accuracy and reliability may be compromised by sensor malfunction or underlying disease. As an example, if one was to use biosensors which analyze ions or solutes within a body fluid then an underlying disease process may alter the fluid composition in a manner which decreases the sensitivity and/or specificity of the sensor-derived data. In the case of cerebrospinal fluid (CSF), if one was to utilize sensors which measure protein content in CSF (which is routinely negligible), this would be an effective biosensor strategy unless the patient was to have a disease process (e.g., meningitis) which causes elevated protein levels in CSF.

In another embodiment of the present invention, computer learning and artificial intelligence (e.g., neural networks) are used to facilitate rapid data analysis and improved understanding, which reduces reliance on human error, which may be of particular concern given the stress induced environment associated with medical device placement. A neurosurgeon tasked with placement of a ventriculostomy tube in the brain has to deal with potentially life-threatening complications, and as a result, should not divert his/her focus on extraneous data.

Collectively, the above embodiments of the present invention can be applied to any medical device and organ system/anatomy to improve device placement and functionality.

In one embodiment, as listed in Table 1, there are several different classes of biosensors 101, 102 which can be embedded within the different types of medical devices—the selection of which is influenced by underlying anatomy, functionality, and device structure. As the number, functionality, and performance of bio sensors and nanotechnology continues to advance, the list will expand, and the list in Table 1 is not limited thereto, but merely reflects relevant examples of the present invention.

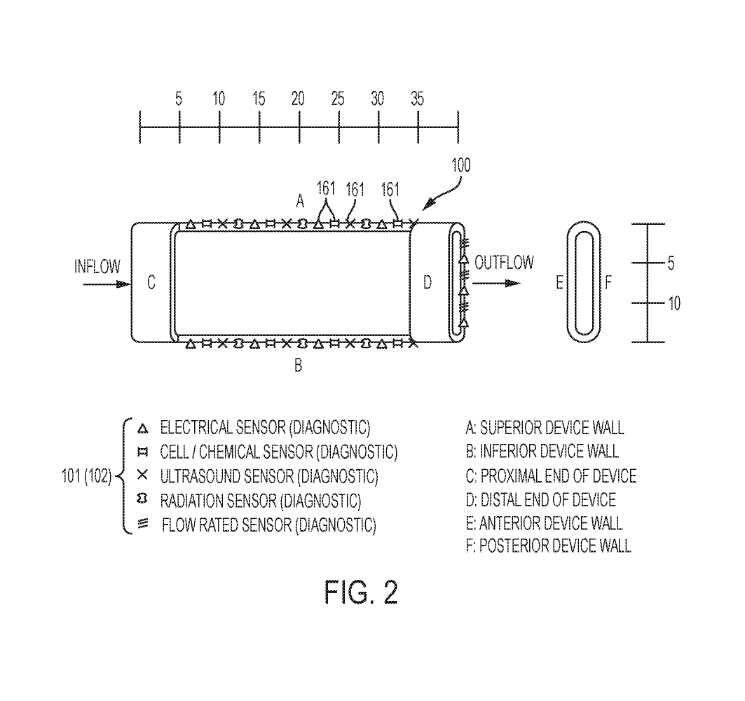

In one embodiment, one category of biosensors that are used for medical device 100 localization are chemical biosensors (see FIG. 2), which can measure organic solutes, chemical compounds, and various ions. The success of these biosensors 101, 102 in device localization is predicated on the fact that the chemicals these biosensors quantify are either extremely high or extremely low in quantity relative to comparable levels found in adjacent anatomic structures.

One example of chemical biosensors, would be those sensors which measure fluid protein levels. In CSF, normal protein levels are extremely low while in abscess cavities, protein levels are extremely high. If one is inserting a ventriculostomy tube with the goal of final positioning within the ventricular system of the brain, the determination of extremely low fluid protein would be indirect evidence of correct placement. On the other hand, if an operator is inserting a percutaneous drainage tube into an abscess cavity, the measurement of high protein levels would be indirect evidence to confirm placement within the abscess cavity. One example where measurement of organic solutes can be used for device localization (e.g., bladder catheter, nephrostomy tube) would be where biosensors measure urea and/or creatinine levels, due to their high levels within urine.

In another embodiment, another category of biosensors is tasked with cellular identification and measurement, which can be of value in both pathologic and non-pathologic states. In the normal (i.e., non-pathologic state), biosensors which can identify red blood cells and platelets are valuable for localizing a medical device in blood vessels, whereas in pathologic states, the ability to detect localized congregation of white blood cells are an indirect marker for inflammation or infection.

In another embodiment, electrical biosensors (see FIG. 2) are another type of sensors which can be helpful in device localization. Of particular note is the heart where physiologic electric activity is prevalent. In the example of a cardiac pacemaker or automated defibrillator, the device 100 typically passes from the superior vena cava and into the various cardiac chambers, each of which has its own characteristic electrical activity, which can be identified through electric biosensors 101, 102.

In one embodiment, medical device localization within vascular anatomy can be assessed in a number of ways—the most important of which is program-analyzed flow characteristics including (but not limited to) flow velocity, directionality, and pressure. In addition, the luminal diameter of the corresponding blood vessel is a helpful data point when placing an intravascular catheter, since smaller dimeter vessels are often fraught with complications such as catheter occlusion and venous thrombosis.

By having the ability to simultaneously measure vascular flow and luminal measurements throughout the course of catheter placement, the technical success of the procedure is enhanced along with minimizing complications (e.g., vascular injury, bleeding, malpositioning). These real-time vascular measurements can also assist in defining adjacent vascular anatomy during the course of device placement.

As an example, suppose a physician is inserting a central venous catheter 100 into the right subclavian vein, with the goal of positioning its distal end at the cavoatrial junction. As this venous catheter is advanced, it will encounter a number of branch vessels including the right internal jugular vein, innominate vein, and numerous smaller venous tributaries. If the catheter 100 being inserted was to deviate from its intended course into one of these vessels, it could have associated adverse consequences.

However, with the present invention, the malpositioning of this catheter 100 can be avoided through continuous flow and luminal data from the sensors 101, 102, with analysis by the program, while also providing real-time identification of the exact location of the venous catheter 100 at any point in time. As the catheter 100 reaches the junction of the right subclavian and internal jugular veins, the characteristic downward flow of the right internal jugular vein will be identified. If on the other hand, the catheter was to inadvertently cross the midline into the innominate vein, it would encounter the left to right flow of the innominate vein. Upon recognizing these characteristic flow patterns, the operator would be aware of the specific catheter 100 location, and be cognizant if the catheter 100 was to unintentionally be advanced into either one of these venous structures.

At the same time, if the operator was to mistakenly advance the catheter 100 into a small venous tributary, the biosensor derived data would show an immediate decrease in flow velocity and luminal diameter, and the program would alert the operator by various electronic methods (i.e., warning on the computer screen, sound, etc.) of the abnormal position before a complication may occur. If the catheter 100 was to be advanced into the right atrium (which is just beyond the intended termination point of the cavoatrial junction), the enlarged luminal diameter of the right atrium would be immediately recorded by the program in the database, along with the lack of antegrade vascular flow. In essence, the biosensor-derived data can provide a real-time anatomic roadmap to the operator to facilitate catheter placement.

In one embodiment, similar biosensor-derived flow, pressure, and diameter measurements can also be applied by the program to device 100 localization within the respiratory tract (i.e., pulmonary airways), as in the case of endotracheal tube placement. The airflow patterns and pressure measurements within the airways, as analyzed by the program, can provide operator guidance as the endotracheal tube is inserted, firstly, to ensure adequate placement in the trachea, and secondly, to ensure the endotracheal tube has not been advanced in either of the main bronchi. Pressure measurements can also provide guidance in the setting of thoracotomy tube placement, which entails placement of a catheter or tube 100 into the pleural space of the thorax for the purpose of either air or fluid evacuation form the pleural space. Additional oxygen (O₂) or carbon dioxide (CO₂) sensors embedded in the thoracotomy tube can assist in ensuring that the thoracostomy tube has not been advanced into the lung parenchyma, while also evaluating for the possibility of a bronchopleural fistula.

In one embodiment, an important biosensor used for device 100 localization is ultrasound (see FIG. 2), which can be used in a number of different medical devices for anatomic guidance. The versatility and myriad of device localization data which ultrasound sensors can provide includes (but is not limited to) flow, volume, pressure, fluid characteristics, cellularity, size, and motion. While this data is especially well suited for vascular applications (e.g., arterial or venous placement), it can also be applied to other medical devices as well; in both physiologic and pathologic conditions.

In one exemplary embodiment of physiologic ultrasound guidance and localization, is an intrauterine device (IUD) which is inserted into the endometrial cavity of the uterus for the purpose of contraception. Ultrasound capability for the device 100, can assist in device 100 placement by outlining the confines of the endometrial cavity to the provider, providing measurements related to the depth of the device 100 within the endometrial cavity, identifying underlying pathology which may adversely affect device function and/or positioning (e.g., endometrial polyp, submucosal polyp), and delineating boundaries with adjacent anatomy (e.g., myometrium).

In addition to its anatomic localization properties during device 100 placement, ultrasound sensors 101, 102 also have the ability to continuously monitor device positioning throughout the duration of the device 100, which in the setting of IUD placement often encompasses several years. In the event that the device was to migrate and become a potential health hazard (e.g., perforation of the uterine wall), ultrasound sensor data can provide important “early warning” signs, which can assist in device 100 location adjustment before significant injury was to occur. This proactive role of ultrasound identifies the potential for improved device 100 performance on a continuous day-in, day-out basis, without the requirement for proactive intervention of ancillary testing on the part of clinical providers.

In one exemplary embodiment of where ultrasound sensors 101, 102 can guide device 100 placement and provide continuous monitoring for pathologic conditions, is in the placement of a drainage catheter 100 in the setting of a pathologic fluid collection (e.g., abscess, hematoma). In this setting, a drainage catheter 100 is frequently inserted for diagnostic and/or therapeutic purposes, and often left in for the purpose of continuous drainage until the pathologic collection has completely resolved. The ability to embed miniature ultrasound sensors 101, 102 throughout the entire length of the device 100 provides for continuous analysis of the device 100 position relative to the collection, any change in device 100 positioning, and interval change in the fluid collection itself, both in volume and internal composition.

Along the same lines, in another exemplary embodiment, ultrasound sensors 100 can be embedded within the outer walls 104 of an intra-arterial stent graft 100 for the purpose of continuous assessment of device positioning (relative to the arterial anatomy), as well as analyzing underlying pathology. In the example of an abdominal aortic stent graft used to treat an abdominal aortic aneurysm, the embedded ultrasound sensors 101 can simultaneously assess device positioning while also evaluating for pathologic change in the underlying aneurysm (e.g., leakage, expansion in size). Additional ultrasound sensors 102 embedded within the inner walls 103 of the device 100 can also assess stent patency which is a fundamental component of device 100 functionality. This illustrates how device-embedded ultrasound sensors 101, 102 can perform several unique functions (i.e., device positioning, device functionality, assessment of underlying pathology), through the continuous creation, recording, and analysis of objective sensor-derived data.

While additional examples can be presented, it is clear that the above-discussed examples of the present invention demonstrate the diversity of available (and evolving) biosensors 101, 102 and how they can be embedded within various types of medical devices 100 to create an objective methodology for real-time and continuous assessment of anatomic positioning, both in physiologic and pathologic conditions. As biosensors and medical device innovation continues in the future, the applications of the invention will also expand and continue to evolve. At some point in time, it will be possible to create self-navigating medical devices based upon the combination of biosensor derived real-time data, computerized learning, patient specific anatomic reference maps (which will be subsequently discussed), and development of device self-propelling technologies.

Internal Vs External Device Localization

In U.S. patent application Ser. No. 15/257,208 (incorporated by reference), the disclosure describes how biosensors and nanotechnology directly embedded in medical devices can be used to guide device placement and anatomic localization in accordance with biosensor-derived standardized data measurements.

In another embodiment, anatomic localization of medical devices can utilize incorporation of ultrasound sensors and/or metallic markers into the medical device. In the “internal” mode of operation, the ultrasound sensors 101, 102 embedded in the device 100 would obtain and communicate ultrasound images through wireless transmission from the sensors 101, 102 to the computer system 106 and any handheld devices 105, to inform the operator, to assist in defining the device 100 position within the body. This would be particularly useful for fluid filled anatomic structures (e.g., bladder, stomach, blood vessels), since fluid facilitates transmission of ultrasound waves.

In the previous example of a nasogastric tube, the addition of device-embedded ultrasound sensors 101, 102 provides complementary data to the physiologic biosensor data (e.g., pH, CO₂) to enhance device localization through combined “physiologic” and “anatomic” data. As the nasogastric tube passes through sequential anatomic structures (e.g., esophagus, stomach, and duodenum), device 100 localization can be defined through both comparative pH measurements, as well as differences in anatomy (e.g., luminal size, wall thickness, internal flow characteristics). This added “anatomic” data can prove to be particularly beneficial when unexpected pathology is present or when localization is limited by physiologic data alone.

As an example, if a patient had a hiatal hernia or experienced gastroesophageal reflux, then pH measures derived from biosensor 101, 102 readings when the device 100 was situated in the distal esophagus and/or hernia sac might mistakenly be interpreted as the device 100 being located in the stomach. Data analysis by the program would yield several clues to correct this misinterpretation, such as correlating the length of device 100 passage from its insertion point with the change in pH measures, the lack of data consistency over time, and correlation with historical patient data. Since well-defined measures of device 100 passage would be well established through continuous device 100 data collection and analysis of the data by the program, one would realize that the typical length from the insertion point (e.g., nasal cavity) to the proximal stomach is 25-30 cm, and if the abnormal pH reading occurred at 20 cm, this would be suggestive of underlying pathology.

Secondly, in the case of gastroesophageal reflux, the pH measurements fluctuate over time, commensurate with each episode of abnormal reflux of gastric acid into the esophagus. As data is continuously recorded by the program into the database, the “up and down” nature of the recorded pH measurements would be indirect evidence to support the diagnosis of reflux and indicate the device location in the esophagus and not the stomach.

Thirdly, historical analysis of the individual patient's device and/or medical databases by the program, would provide knowledge of the presence of pre-existing pathology. In the case of pre-existing pathology (e.g., hiatal hernia, reflux), the diagnoses may be established through prior imaging tests (e.g., CT scan, upper GI series), clinical tests (e.g., endoscopy, manometry), or previous medical device data. This illustrates an important application of the present invention discussed below—namely, that the real-time data obtained by current device measurements can be correlated by the program with historical device and patient clinical data to improve data analysis and understanding. This data mining of historical device and clinical databases can be automated and performed by the program prior to performance of the current device 100 placement and presented (i.e., on computer screen) to the clinical operator (e.g., nurse, physician) prior to initiation of the procedure, along with expected data variations to be encountered, which are separate from “customary” data measurements. In the example of a hiatal hernia detected on a previous abdominal CT exam, the computerized analysis and decision support features from combined mining of the CT report and patient specific device database could include the following data:

1. Presence of pathology or anatomic variation: Hiatal Hernia (Abdominal CT 12/2/15).

2. Size of Hernia: 6.2×4.1×5.0 cm.

3. Expected location of abnormality: 21 cm (from nasal cavity to origin of hernia).

4. Expected location of Stomach: 27 cm (from nasal cavity to proximal stomach).

5. Expected location of Duodenum: 41 cm (from nasal cavity to proximal duodenum).

6. Expected pH measurements.

A. Esophagus: 7-9.

B. Hiatal hernia: 4-6.

C. Stomach: 4-6.

D. Duodenum: 8-10.

With this data, the clinical operator would have knowledge as to the presence of pathology which may affect device placement, pertinent characteristics of the pathology in question (e.g., size), the expected anatomic landmarks to identify during device advancement and their specific locations relative to the device entry point, and the biosensor-derived measurements specific to these different anatomic locations.

Returning to ultrasound guidance, the ultrasound sensors 101, 102 embedded in the device 100 provide a secondary source of device 100 localization, which can be used to supplement biosensor-derived physiologic data measurements. The ultrasound images obtained would be transmitted via wireless communication networks from the device 100 to the computer system 106, in the same manner the biosensor data is communicated (see U.S. patent application Ser. No. 15/434,783 (incorporated by reference)).

In this exemplary embodiment, as the device 100 is advanced from the esophagus to the stomach, one would be able to visualize anatomic structural differences between these two different regions based upon size, wall thickness, and contained fluid. When the position markers for these anatomic transition points are correlated by the program with the position markers for physiologic transition points, an added degree of accuracy in device localization is provided. At the same time, when an unexpected anatomic variation or pathology is encountered, the ability of the program to correlate physiologic and anatomic data can prove valuable.

In the example of the hiatal hernia, suppose no pre-existing knowledge of the hernia is available at the time of device placement. The unexpected drop in pH levels at lower than expected positional measurements might lead the operator to think the device has entered the stomach when in actuality it is within the hernia sac. Having the ability to correlate these pH measures with ultrasound images allows the operator to identify the true cause of the pH/position discrepancy (since a hiatal hernia has a fairly straightforward anatomic appearance when compared with the neighboring esophagus and stomach).

In another exemplary embodiment, in addition to “internal” ultrasound guidance to device localization, ultrasound sensors 102 can also be used for “external” device 100 localization. In this application, ultrasound location markers are embedded within the medical device 100 and are used in concert with externally positioned ultrasound sensors 101 at various locations on the body surface. In this embodiment, ultrasound markers 102 are embedded at predefined positions within the medical device and serve as “acoustic reflectors” of ultrasound waves which are transmitted from ultrasound probes positioned on the body surface. Once these externally emitted ultrasound waves come in contact with the device-embedded acoustic reflectors, an acoustic shadow is seen and the depth and location of this acoustic shadow can be recorded in the device database by the program, along with the specific external locations of each superficially located ultrasound probe. Using triangulation, the various echo patterns from each superficially located ultrasound probe and the internal device 100 can by analyzed by the program to provide a three-dimensional location of the device 100. Since multiple “acoustic reflectors” can be embedded at various predefined positions within the device 100, the entire length of the device 100 can be determined relative to the internal anatomy in which it resides. This may prove especially valuable when a given medical device 100 transcends multiple anatomic points, or a portion of a device 100 has been broken and becomes detached from the native device 100.

In one exemplary embodiment, to illustrate a medical device 100 crossing anatomic boundaries, one can use the example of a gastrostomy tube 100 which is partly positioned inside the stomach and partly outside of the stomach. In the example of feeding and/or drainage tubes, it is important to ascertain the specific locations of all individual device orifices, since they are important to device functionality as well as potential device-related complications. If an individual orifice of the device 100 lies external to the anatomic structure of interest, the potential exists for this orifice to result in fluid egress outside of the desired location. If a feeding tube has three orifices (i.e., holes) used for fluid passage, and two of those are within the desired location (e.g., stomach), while one is outside of the desired location (e.g., peritoneal cavity), then fluid passing through the tube will exit all three holes, including the malposition hole in the peritoneal cavity (which can lead to peritonitis, a life-threatening condition).

By embedding these acoustic reflectors 102 at a series of predefined locations in the device, one can determine the entire location of the device 100 as well as its individual components. In the example of the gastrostomy tube 100 with one hole outside of the stomach, the corresponding acoustic reflector 102 at this location can be closely monitored and re-evaluated by the program for any device manipulation (e.g., advancement). In the event that further verification of orifice placement is required, an echogenic fluid could be injected and localized as it passes from each of the individual orifices. Additional placement of ultrasound sensors 102 in the device 100 (e.g., adjacent to the device orifices), can provide additional visualization as fluid exits the orifice and accumulates in the adjacent anatomy (e.g., gastric lumen).

In one exemplary embodiment of a broken medical device (e.g., sheared vascular catheter), the broken device component may migrate and be positioned in an unusual/unexpected location. By utilizing device-embedded acoustic reflectors 102 and externally located ultrasound probes 101, the broken device can be localized using triangulation, by the program. In addition, if a closed retrieval process is attempted (e.g., endoscopically), ultrasound embedded sensors 101, 102 in the retrieval device can be used to track and located the device 100 through its embedded acoustic reflectors.

Inadvertent and Unexpected Device Repositioning

To follow up on this issue of device 100 migration, depending upon the specific type of device, anatomic location, and functionality, changes in device 100 positioning can have different degrees of clinical significance. A nasogastric tube 100 which is placed in the stomach may change positioning by several centimeters before any clinical ramifications occur. On the other hand, movement of an arterial stent 100 in a relatively small arterial structure (e.g., renal or intracerebral artery), can have significant clinical consequences.

For this reason, it is important to continuously monitor and assess device 100 positioning after initial placement has been successfully performed. In conventional practice, routine surveillance of device positioning is performed with medical imaging exams (e.g., x-ray, CT), but this requires proactive action on the part of the clinical provider and is often performed on a periodic and intermittent basis. In addition, this incurs radiation exposure to the patient along with considerable cost to the healthcare payer.

In contrast, the present invention discloses a far superior approach to the conventional methods, and the program continuously monitors the device 100 position, and creates the ability to automatically report this data (via electronic methods, such as a warning on the computer system, text, email, etc.) to the clinical provider in accordance with predefined rules, specific to the individual type of device, provider preferences, and defined best practice guidelines. Since the clinical impact of device movement is not only determined by the actual change in distance, but also by its starting point, the analysis of device positional change is a dynamic process.

As an example, a nasogastric tube which originally terminates in the distal stomach and has now been pulled back 4 cm (into the middle portion of the stomach), will have no significant impact on device functionality or patient safety. If the same nasogastric tube 100 was originally inserted into the duodenal bulb (i.e., proximal duodenum), the same device positional change of 4 cm will now place the device 100 in the distal stomach, which may be considered undesirable by the clinical provider, if they specifically wanted the nasogastric tube to be duodenal in location. The net effect is that device 100 positional change is a dynamic process, the analysis of which is in part dependent upon individual clinical providers' preferences and desired functionality. The computer system can be programmed to accomplish all these actions based on these predetermined preferences, the desired functionality, etc.

The ability to continuously record, monitor, and analyze device 100 positional change using embedded biosensors 101, 102 is dependent upon fixed anatomic reference points. In the case of devices 100 which are externally placed (e.g., vascular catheter, nasogastric tube), the anatomic reference point used to assess device positional change can include the device entry point. For example, the nasogastric tube 100 inserted via the nasal cavity can use the adjacent nasal orifice as the anatomic reference point, with the device sensor 101 of closest proximity serving as the measurement point of interest.

As an example, if the closest sensor 101 to the nasal orifice is located at position 40 cm of the nasogastric tube (i.e., 40 cm from the proximal end of the nasogastric tube), ongoing measurements can be performed delineating the distance between the sensor of record (i.e., at 40 cm) and the fixed anatomic reference point (i.e., nasal orifice). Since these distance measurements can be bidirectional (i.e., forward or backward movement of the nasogastric tube), the measurements are recorded as either positive or negative numbers. This provides an easy way to understand positional change measurement. Since the analysis of these positional changes is dynamic and in part dependent upon individual provider preferences, the reporting of these measurements can be customized in accordance with predefined rules, which can be determined by an individual provider, the healthcare institution of record, or professional guidelines (i.e., best practice standards).

In the case of medical devices 100 which are internally placed (i.e., via surgery or endoscopy), the fixed anatomic reference point would include an “internal” structure, in which a sensor 101 is placed for the purpose of providing continuous distance measurements between it and the designated device biosensor 101 of record. As an example, suppose an internal biliary stent 100 is placed (through either surgery or endoscopy), in order to provide biliary drainage in the setting of common bile duct obstruction due to pancreatic head cancer. At the time of stent placement, the surgeon positions a biodegradable sensor 101 in the pancreatic head adjacent to the ampulla of Vater (i.e., point where common bile and pancreatic ducts originate) to serve as the fixed anatomic reference point. When correlating with the device sensor roadmap, it is determined that the device biosensor 101 in closest proximity to this anatomic reference point is located at device positon 1.2 cm. As a result, all subsequent device positional measurements will use this 1.2 cm as the reference point of initial device placement and subsequent device positional changes. At the time of completion of the device placement, the surgeon can manually input the range of “acceptable” device position or defer to a predefined default range, which has been defined in accordance with the type of device, anatomy, clinical indication, and device functionality. If the predefined “acceptable” positional change is 1.0 cm, then any positional change measurement in excess of 1.0 cm (either positive or negative) as determined by the program, will automatically trigger an alert (via electronic methods etc.) to the corresponding medical staff tasked with corresponding patient care.

In addition to the intermittent minor positional changes which regularly occur, unexpected more dramatic positional changes can take place in device positioning, which are often the result of intentional or inadvertent human acts. An intentional (and commonly experienced) act may include the patient attempting to pull the device out; which, if successful, requires reinsertion of the device along with some sort of additional preventative measures to prevent reoccurrence of intentional device removal.

In another example of inadvertent acts, the clinical staff (e.g., nurse) responsible for device routine maintenance may unintentionally have contact with the device causing significant positional change. While both scenarios will result in automatic calculation of device positional change and automated alerts to authorized recipients, by the program, in the event that the predefined positional threshold is exceeded, this information may often be too late to have appositive impact, since the device may have been repositioned to such a degree (or even removed) to not allow for repositioning of the same device as a relevant option. Instead, the device would have to be reinserted, with the procedure having to begin from the start, all over again.

In one embodiment, the present invention includes a proactive interventional strategy to overcome these disadvantages, by placing biosensors 101 on the external portion of the device 100, the biosensors 101 which are specifically designed to detect motion and/or pressure in close proximity (see FIG. 2). In the event that a patient or clinical care provider was to brush up against these external motion/pressure sensors 101, the action and its severity will be recorded by the program along with the specific location of the involved sensors. Since minor points of contact will frequently take place, the continuous measurements of these sensors by the program, will provide a baseline for differentiating between “minor” and “major” contacts, as well as the expected frequency and specific timing of contacts.

In one embodiment, the timing of these events becomes important in helping to differentiate between “inadvertent” and “intentional” acts; inadvertent acts may occur during the time clinical staff is performing routine tasks (e.g., nasogastric tube feedings or flushes), whereas intentional acts may occur at unexpected times (e.g., middle of the night when the patient becomes agitated).

In one embodiment, the ability of the present invention to continuously record and analyze individual biosensor data within the medical device, provides an important tool to identify unexpected measurements in a specific device location. In the event that a device was partially pulled out from its original location, the resulting biosensor-derived data would reflect a sudden change in measurements, specifically to those biosensors located in the portion of the device which is now located outside of the target location. In the example of a nasogastric tube in which biosensors continuously measure pH levels, biosensors which were originally located in the stomach and are now located in the esophagus will record new pH levels reflective of this positional change. The location and number of biosensors which have been repositioned in the esophagus will alert the provider as to the exact distance and time of positioning change. By incorporating artificial intelligence techniques (e.g., machine learning) and automated statistical analysis of the continuous biosensor derived data, automated alerts can be generated by the program to authorized providers at the exact time the event takes place, along with the accompanying data. In addition, the device can have an integrated alarm system which is activated whenever a predefined threshold is identified, which provides an auditory alert, which can serve as a deterrent to intentional acts of adverse device positional change.

When these motion/pressure measurements are continuously recorded and analyzed by the program, a device and patient specific profile can by prepared by the program, which may assist in determining interventional requirements. In the example of a nurse who frequently causes higher than expected positional changes during routine care, additional mentoring and/or training in device care may be required. On the other hand, a patient who frequently causes device positional change due to noncompliance or excessive anxiety, may require additional sedation or physical restraints, which can be customized in accordance with the specific times of the day in which these intentional actions take place. As new device features are introduced which aim to reduce device positional change, the ability to perform comparative device analysis provides an effective and powerful method of assessing device performance and impact of the intervention (i.e., before and after analysis of device positional data).

Anatomic Reference Maps

In one embodiment, while the embedded biosensors can independently be used to localize positioning of the medical device and establish important anatomic landmarks within the human body, the biosensor-derived data can be synchronized with other anatomic data to create a patient-specific anatomic reference map. A variety of anatomy visualization tools and technology can be used by the program to create this map, including (but not limited to) cross section medical imaging tests (e.g., CT, MRI, ultrasound), endoscopic technologies (e.g., bronchoscopy, colonoscopy, cystoscopy), or photography.

One example of how these patient specific anatomic reference maps can be created is through the use of CT data, which is commonly performed in a variety of organ systems for medical diagnosis. In the example of an abdominal/pelvic CT exam, cross-sectional anatomic data is acquired from the level of the inferior thorax to the inferior pelvis, containing all organ system anatomy within the regions of coverage. The anatomic data contained within this area of coverage can be further subdivided through image segmentation (i.e., an established from of image processing) to isolate a single organ system (e.g., gastrointestinal tract, genitourinary system) of interest, with the corresponding data used by the program to create 2 or 3-dimentional anatomic maps. If one desired to extend the field of interest, data from separate CT exams (e.g., chest CT, neck CT) can be used to expand the purview of anatomic coverage for the specific organ system of interest. In the case of the gastrointestinal (GI) tract, combining data from neck, chest, and abdominal/pelvic Ct exams would in effect create an anatomic reference map extending throughout the entire length of the GI tract, from the nasal cavity to the anus.

In one exemplary embodiment, if a clinical provider wanted to review the anatomic reference map prior to placement of a medical device specific to the GI tract (e.g., nasogastric tube, gastrostomy tube, jejunostomy tube), they could retrieve the GI anatomic reference map from the patient database, and review either 2 or 3-dimensional anatomic data specific to the patient and medical device of interest. Since the anatomic data would be specific to the individual patient, all corresponding anatomic variation of the individual patient's GI system would be reviewable and analyzable by an authorized healthcare provider including (but not limited to) congenital anatomic variation (e.g., intestinal malrotation), surgical changes to anatomy (e.g., gastric bypass), and organ system related pathology (e.g., hiatal hernia).

In one embodiment, the anatomic reference map data would include measurements of key anatomic landmarks which are pertinent to the specific anatomy and medical device of interest. In the example of a nasogastric tube which is routinely inserted in the nasal cavity and terminates in the stomach or duodenum; all intervening anatomic structures (e.g., nasopharynx, oropharynx, esophagus) would be included in the display and program analysis, along with corresponding anatomic data and measurements.

As an example, if a clinical provider wanted to consult the patient specific anatomic reference map prior to insertion of a nasogastric tube, they could request the specific method of display presentation (e.g., 2-dimensional coronal plane) along with distance measurements at specific points of anatomic interest (e.g., origin of stomach and duodenum); inclusive of superposed pathology (e.g., hiatal hernia). By doing so, the provider would be presented by the program with the following exemplary patient-specific anatomic and pathology specific data referable to the gastrointestinal tract.

Insertion Site: Nasal Cavity (Right)

Desired Termination Site: Duodenal Bulb

Intervening Anatomic Landmarks and Distances from Nasal Cavity Insertion Site:

A. Origin of Esophagus 8.5 cm

B. Origin of Stomach: 20.4 cm

C. Origin of Duodenum: 33.6 cm

D. Length of Esophagus: 11.9 cm

E. Length of Stomach: 12.2 cm

F. Anatomic Variants: None

G. Pertinent Surgery: Nissan Fundoplication: 18.8 cm

H. Pertinent Pathology:

1. Nasal Polyps (Left Nasal Cavity): 2.2 cm

2. Hiatal Hernia: 19.0 cm

3. Duodenal Ulcer: 35.1 cm

This exemplary data can be customized by the program to the specific needs and preferences of the individual provider (i.e., customizable anatomic reference maps), and can incorporate pertinent clinical and anatomic data from a variety of data sources contained within the patient electronic medical record (e.g., medical imaging studies and reports, operative notes, history and physical exam, pharmacy records). In the example provided, the designated insertion site is specified as the right nasal cavity due to the presence of previously diagnosed left nasal cavity polyps. The presence of other pathologies along the course of anatomic interest (e.g., hiatal hernia, duodenal ulcer) is also provided by the program, along with their respective longitudinal distances to provide prospective guidance to the provider prior to performance of device insertion, which would be of interest in medical device selection and procedural strategy.

In one embodiment, in addition to end-user customization, the present invention can also be customized specific to the technology being used. Since devices from different manufacturers differ in design, size, and functionality, pre-placement planning should ideally take these often, subtle device differences into account. As an example, if one type of nasogastric tube has greater flexibility (i.e., less rigidity) than a competitor's model of nasogastric tube, it may be shown to exert a greater tendency to fold onto itself (i.e., coil) at a characteristic location (e.g., proximal stomach), which prevents optimal nasogastric tube passage into the distal stomach and/or duodenum. With this knowledge in hand prior to device placement, the operator (e.g., physician, nurse) may be presented by the program with the options of: selecting a different type of device with less preponderance to coil (i.e., if readily available); proceeding with the device already selected; or modifying the placement strategy in keeping with the newly acquired knowledge.

One example of modifying the placement strategy might be to use an optional removable insert in the device to provide greater rigidity during the placement process to reduce the chance of tube coiling and improve the passage of the tube into the desired location (e.g., proximal duodenum). Once successful placement is accomplished, the insert can be removed and the device allowed to operate in its native form.

In one embodiment, the present invention can incorporate patient specific anatomic data into the device prior to placement with the purpose of providing visual or auditory feedback during the course of the procedure, as predefined landmarks/distances are realized. In one example, the physician or nurse tasked with placement of the nasogastric tube may input a request in the computer system, for an auditory prompt once the device has traveled 20.4 cm. This important anatomic landmark/distance should signal device entry into the stomach, which should be accompanied by a dramatic decrease in pH, which will be determined by pH sensor measurements run by the program. In the event that the 20.4 cm alert is not accompanied by a dramatic drop in measured pH by the biosensors, then the program can alert the provider (via electronic methods) to either malpositioning of the nasogastric tube, unexpected change in patient anatomy, calculation error in the anatomic reference data, or malfunctioning of the biosensors embedded in the device walls.

In one embodiment, by synchronizing biosensor and anatomic reference map data, the program can make real-time modifications to the patient specific anatomic reference map data. If for example, the lead biosensor drop in pH is first detected at 21.0 cm (and not the expected 20.4 cm), then the modified measurement for “origin of the stomach” is now corrected by the program to 21.0 cm from the original 20.4 cm. This ability of the present invention to synchronize data is important for future analysis and device placements, to provide continuously updated data and analyses for future providers and device placements.

An alternative example may include the program recording a drop in pH measures at an expectant length of 17.2 cm (from the device insertion point). Since the hiatal hernia does not originate until a device length of 19.0 cm (based upon predefined anatomic reference map data), that would not explain the derived pH measurement. An alternate explanation would be due to gastroesophageal reflux (in which gastric acid flows in a retrograde fashion into the esophagus), which would explain the lower than expected pH measurements in the distal esophagus. One easy way to confirm this as the underlying etiology would be to stop advancing the nasogastric tube at this level and have the program record a series of pH measurements. By doing so, if the pH measures fluctuate (as opposed to being constant) then this would confirm the presence of gastroesophageal reflux which occurs intermittently. Fixed or stationary anatomy (e.g., hiatal hernia, or gastric diverticulum) would be expected to provide consistent pH levels which do not deviate over time. The new result is that the ability of the program of the present invention, to correlate and synchronize biosensor and anatomic data into a single analysis tool, provides a method of verifying and updating both anatomic and pathologic data, which can serve as an important tool for optimizing medical device placement in real time and making requisite adjustments at the point of care.

In one embodiment, the patient specific anatomic reference map data can also be correlated by the program with the device-specific sensor roadmaps to provide an anatomic reference point for each individual sensor contained within the medical device. While linear unidirectional medical devices such as a nasogastric tube have a fairly straightforward distribution of sensors, which are easily tracked relative to the anatomic position within a specific organ system, complex multi-directional medical devices like an inferior vena cava (IVC) filter or bifurcated arterial stent graft, have individual device components and associated embedded sensors distributed in different directions and potentially different anatomic structures.

As an example, an IVC filter may be shaped like an umbrella with multiple spokes originating form a common focus and these individual struts may have perpendicularly oriented ends which are designed to attach to the IVC wall. A bifurcated arterial stent graft in the abdominal aorta will have one portion of the stent extending into the right common iliac artery and another into the left common iliac artery. In addition, it may have an outer component contiguous with the native aorta wall and inner components contained within a central lumen.

The net result is that medical device design and structure is often complex, and as a result, the individually embedded sensors may have similarly unusual positioning and orientations. Each device-specific roadmap provides a schematic representation of each individual sensor which can be directly localized relative to its adjacent anatomy by the program correlating the patient specific anatomic reference and device specific sensor roadmaps. This takes on heightened importance when an individual sensor malfunctions or becomes disabled, requiring correction of the sensor-derived data and analyses, or when an individual component of the device becomes damaged.

A relevant example of how individual sensor-derived data within a single device requires specific correlation with anatomy can be illustrated with an intravenous catheter which is inserted in the right internal jugular (LI) vein, extends into the superior vena cava (SVC), into the right atrium and ventricle, and terminates in the inferior vena cava (IVC). A sensor embedded in the device at the level of the SVC measuring flow directionality shows flow moving in a downward direction (i.e., from the neck to the heart), while sensors embedded in the portion of the device in the IVC, shows flow oriented in an upward direction (i.e., from the abdomen to the heart. In both cases, the flow is correctly oriented (i.e., towards the heart), but depending upon its anatomic location, the sensor-derived flow measures show directionality in opposite directions. If the specific location of these individual sensors (within a single device) were not accurately correlated by the program with their specific (and individual) anatomic location, one may mistakenly interpret the data as showing abnormal flow reversal in one component of the device.

In one embodiment, in the setting where a medical imaging exam (e.g., CT) or visualization procedure (e.g., endoscopy) is performed with a medical device in place, the clinical provider would have the opportunity to further analyze device location with anatomy; but not readily visualize individual device embedded sensors due to their small size which is beyond traditional visual discrimination. In one embodiment, devices may have the capability to emit a characteristic signal for the purpose of identification, which in turn can be used to localize relative to adjacent anatomy or pathology.

As an example, suppose a patient has undergone surgical fixation of a hip fracture and the orthopedic surgeon is concerned about delayed healing and abnormal motion at the fracture site. While biosensors are embedded throughout the length of the fixation device, the data of greatest clinical importance is from those sensors specifically localized along the fracture margins. Since fractures are frequently communized and complex in orientation, identifying the individual sensors along the fracture margins may be problematic and often difficult to accurately localize in fine detail. While a cross sectional imaging study (e.g., CT) may assist in defining the overall anatomy of the hip and fixation device, it does not provide accurate detail to visualize individual sensors. At the same time, visualization of anatomy and pathology (i.e., fracture margins) may be further compromised by beam hardening artifact caused by the metallic density in the orthopedic device.

The solution would be to have the individual sensors to emit a characteristic signal (e.g., visual, auditory), which can identify its specific location within the device by correlating the sensor-specific signal with the device roadmap. The program roadmap would not only visualize sensor type and distribution within the device, but also provide the characteristic “signature” of each individual sensor for localization purposes, which may be beyond conventional visualization techniques. By identifying and accurately localizing these individual sensors, the corresponding data within these sensors can be localized and analyzed by the program apart from other nearby sensors of lesser clinical relevance.

Graphical Device Displays

In one embodiment of the present invention, the ability to capture prospective medical device positioning data throughout the lifetime of the medical device provides a mechanism to create an historical record of any given medical device within an individual patient's medical history. In addition to review and analysis by the program of this data in text and numerical formats (which are the primary means with which standardized data is recorded in the medical device database), this data can also be displayed in graphical format. The positional data from the original device placement can be supplemented by subsequent positioning data of the device throughout its use, so that the program's graphical display can present the authorized end-user with an easy to comprehend 2-D or 3-D display of the cumulative variation in device positioning from the time it is placed to the time it is removed. Since a medical device can be replaced and/or reinserted (after intended or unintended removal) on multiple occasions of a single medical event (e.g., hospitalization, course of chemotherapy, surgical procedure and associated post-operative care), the multiple device placements of this device can be displayed under the “single medical event” category. As a result, the graphical display of device positional data can be presented by the program using a wide array of search criteria which include the following examples:

1. Device Category

2. Specific Type of Device

3. Clinical Indication

4. Medical Event

5. Anatomy/Organ System

6. Time

7. Device Data Outliers

8. Individual Healthcare Provider

9. Institutional Provider

10. Device Manufacturer

In the example of device display data based upon “device category”, the end-user can have all data specific to a specific category of medical device displayed by the program on a single display presentation format (e.g., nasogastric tube, central venous catheter, cardiac pacemaker). This cumulative device category data can be further restricted by specifying an additional search variable (e.g., time, medical event); which narrows the data analysis and display presentation to the device category of interest coupled with the secondary search criteria.

As an example, suppose a surgeon is interested in reviewing all device data specific to the category “central venous catheters”, during a defined period of time related to an individual patient's hospitalization. The hospitalization began on Jan. 4, 2016 and ended on Jan. 23, 2016. During the entire period of time, the patient had placement of six different venous catheters; four of which are classified as “central venous catheters”, and two of which are classified as “peripheral venous catheters”. Before the computer-generated display is presented to the surgeon by the program, a number of additional questions are posed to the surgeon regarding his/her individual preferences in display presentation along with clarification of the data to be included in the program analysis. The individual end-user display presentation preferences can be used by the program to create a “user-specific display presentation profile”, which utilizes this data to create a standardized default presentation state whenever a specific authorized end-user generates a request for graphical device display.

Examples of these display presentation preferences include (but are not limited to) color, shading, font, 2 vs 3-D, size, anatomy template, annotations, and orientation). In the event that the individual end-user has a profile on record, unless otherwise instructed, the program of the computer system will automatically generate a graphical device display in accordance with the defined search criteria and end-user's documented display preferences. The end-user has the ability to over-ride the profile default data at any time though a manual editing process, or he/she can modify the graphical display “after the fact” though manual manipulation of the display (using computer tools). Based upon this data, the computer-generated graphical device display would show the 3 central venous catheters which were placed during the time period of the defined medical event, with individual device-specific anatomic and clinical data readily available by highlighting the individual device of interest and selecting the data category of specific interest.

In addition to reviewing device data on the basis of “device category”, one can instead review data based on the specific type of medical device. In the previously described example, the patient had three different central venous catheters during the course of the defined medical event. These included a left internal jugular venous catheter, right and left PICC lines, and a Swan-Ganz catheter. By specifying the specific type of device of interest (e.g., PICC line), the end-user can narrow the search and graphical display to a specific type of medical device. In this example, after specifying the specific type of medical device as “PICC line”, the resulting analysis and display would show only the two PICC lines inserted during the course of the hospitalization of interest.

In a similar manner, device display data can be presented by the program on the basis of clinical indication (e.g., disease diagnosis), a specific medical event (e.g., ICU stay), anatomy (e.g., cardiovascular system), time (e.g., Jan. 4, 2016-Jan. 23, 2016), device data outliers (e.g., device malpositioning), individual healthcare provider (e.g., Dr. Harris Smith), institutional provider (e.g., Good Samaritan Medical Center), or device manufacturer (e.g., Medtronic). The data included in the analysis and display by the program, is not restricted to a single data source, but can be combined from multiple data sources, which fulfill the designated search criteria. As an example, suppose during the course of the defined time of interest, the patient was initially hospitalized at on medical institution and subsequently transferred to a second institution. The data from both institutions (contained within the patient electronic medical records), could be combined by the program to create a single “all inclusive” graphical device display.

In one embodiment, more than one category of device may be analyzed and displayed. As an example, suppose an authorized end-user wanted to review a graphical display of all medical devices inserted by an individual healthcare provider over a 5-year period of time. In the course of this analysis, all medical devices attributed to the provider of interest are reviewed, analyzed, and displayed by the program irrespective of the specific category or type of device. If the authorized party reviewing this data wants to further screen this data in accordance with an individual organ system or anatomic region (e.g., cardiovascular system), they can do so by entering the search criteria of interest (i.e., Anatomy/Organ System: Cardiovascular System). By doing so, all medical devices within the cardiovascular system placed by the provider of record over the time frame of interest are displayed by the program on a single graphical format.

In one embodiment, a unique feature of the present invention is the ability to combine and analyze data from multiple patients, device categories, individual healthcare or institutional providers, or device manufactures. Suppose in this example, a quality assurance (QA) review (program-driven or manual review of data) within an individual medical institution identifies an individual healthcare provider with higher than expected complications related to medical device placement. In order to better understand the frequency and anatomic distribution of malpoisitoned medical devices, the authorized individual (e.g., quality assurance specialist) queries the medical device database to retrieve, analyze, and display all malpositioned medical devices at the institution over the defined period of time for the physicians of concern. In doing so, the QA specialist is presented by the program with a graphical display of the malpositioned devices, which are displayed in accordance with the anatomy/organ system of record, the expected device location, and the abnormal location in which they were positioned. This provides an easy-to-comprehend visual display of the overall frequency of malpositioned medical devices, along with the degree of deviation between “normally expected” and “abnormally observed” device locations. For those devices whose locations are the farthest away from the expected locations, the researcher can simply highlight the device of interest and the corresponding device data will be presented by the program. This provides an easy and efficient method for review and analysis of device data outliers. If the researcher now wishes to simultaneously view “device positon outliers” with “normally positioned devices” from this specific physician, they can modify the search criteria to show both categories, and display the different categories in different colored formats, for example, for easy differentiation. This provides the QA specialist with perspective as to the relative frequency of normal versus abnormal device positioning for this individual physician; along with the associated device categories and organ system.

In one embodiment, this same graphical device display can in turn be used for peer review by professional colleagues to determine the severity of the problem and strategies for intervention (e.g., remedial training, mentoring). In addition, the graphical display format can help the physician of record directly view and better understand the “full picture” of his/her procedural difficulties and strategize how these device positioning complications can be avoided in the future. This same type of analysis by the program can also be extended to comparative analysis of individual device manufacturers, in an attempt to quantify which individual devices are prone to higher levels of device malpositioning and the resulting locations.

It should be emphasized that the above-described embodiments of the invention are merely possible examples of implementations set forth for a clear understanding of the principles of the invention. Variations and modifications may be made to the above-described embodiments of the invention without departing from the spirit and principles of the invention. All such modifications and variations are intended to be included herein within the scope of the invention and protected by the following claims.

What is claimed is:

1. A computer-implemented method of determining medical device positional changes within a body of a patient, comprising:
providing a medical device for internal use within the body of the patient during a medical procedure, said medical device having a plurality of sensors disposed at predetermined intervals along a length of said medical device;
receiving data from said sensors on a position of said medical device in the body of the patient, and recording said data into a database of a computer system, performing an analysis of said data using a processor of said computer system;
wherein when said analysis of said data received from said sensors indicate a positional change of said medical device, issuing an alert that said medical device has changed its position.

2. The method of claim 1, wherein said predetermined intervals include a device origination point, a device termination point, and transition points which indicate anatomical transition points.

3. The method of claim 1, wherein said sensors are disposed in at least one of outer walls or inner walls of said medical device.

4. The method of claim 3, wherein said sensors include one or more types of sensors or biomarkers, including at least one of electrical sensors, chemical sensors, ultrasound sensors, motion sensors, or pressure sensors.

5. The method of claim 4, wherein said sensors measure at least one of pH, oxygen, carbon dioxide, radiation, curvature, coiling, motion, pressure, sound, flow volume, velocity and directionality, fluid characteristics, cellularity, or size.

6. The method of claim 2, wherein said anatomical transition points are fixed.

7. The method of claim 6, wherein position markers for said anatomical transition points are correlated with the position markers for physiologic transition points, to provide accuracy in device localization.

8. The method of claim 1, wherein said data is transmitted continuously by said sensors.

9. The method of claim 1, wherein said alert is issued by electronic methods.

10. The method of claim 7, wherein said data is synchronized with other anatomic data to create a patient-specific anatomic reference map.

11. The method of claim 10, wherein said data from said patient-specific anatomic reference map is incorporated into the medical device prior to placement to provide visual or auditory feedback.

12. The method of claim 11, further comprising:
synchronizing said sensors and said data from said patient-specific anatomic reference map to make real-time modifications to said patient-specific anatomic reference map.

13. The method of claim 12, further comprising:
correlating said data from said patient specific anatomic reference map with device-specific sensor roadmaps, to provide an anatomic reference point for each of said sensors contained within the medical device.

14. The method of claim 1, further comprising:
combining and analyzing data from multiple patients, device categories, individual healthcare or institutional providers, or device manufactures.

15. The method of claim 1, wherein each of said sensors emit a characteristic signal to identify its specific location on the medical device; and
wherein said signal is correlated with a device specific roadmap.

16. The method of claim 1, further comprising:
providing a graphical display of said positional change of the medical device.
